(19) United States
(12) Patent Application Publication
(10) Pub. No.: US 2022/0387761 A1
(43) Pub. Date: Dec. 8, 2022

(54) CATHETER ASSEMBLY

(71) Applicant: TERUMO KABUSHIKI KAISHA, Tokyo (JP)

(72) Inventor: Masahiro ISHIDA, Kanagawa (JP)

(73) Assignee: TERUMO KABUSHIKI KAISHA, Tokyo (JP)

(21) Appl. No.: 17/888,048

(22) Filed: Aug. 15, 2022

Related U.S. Application Data
(63) Continuation of application No. PCT/JP2021/005952, filed on Feb. 17, 2021.

(30) Foreign Application Priority Data
Feb. 20, 2020 (JP) 2020-026882

(51) Int. Cl. A61M 25/06; A61M 25/00; A61M 25/09

(52) U.S. Cl. CPC A61M 25/06; A61M 25/0097; A61M 25/09041; A61M 5/158

(57) ABSTRACT

A catheter assembly includes: a catheter; a catheter hub fixed to the catheter; an inner needle inserted through the catheter; a grip fixed to and holding the inner needle; a first support portion, which is a member separate from the grip and is configured to support an outer side of the catheter; and a second support portion, which is a member separate from the grip, has at least a part that is located at a position facing the first support portion, and is configured to support the outer side of the catheter. The first support portion and the second support portion are releasably engaged with each other.

### CROSS-REFERENCE TO RELATED APPLICATIONS

This is a bypass continuation of PCT Application No. PCT/JP2021/005952, filed on Feb. 17, 2021, which claims priority to Japanese Application No. JP2020-026882, filed on Feb. 20, 2020. The contents of these applications are hereby incorporated by reference in their entireties.

### BACKGROUND

The present disclosure relates to a catheter assembly having a structure capable of supporting the outer side of a catheter at the time of puncture with the catheter and an inner needle.

When an introduction unit for infusion, blood transfusion, or the like is constructed in a treatment target (patient), a catheter assembly as disclosed in US 2016/0256667 A is used. The catheter assembly includes a multi-structure needle in which an inner needle is inserted into a catheter (outer needle). When the catheter assembly is used, a user punctures the multi-structure needle into a body of the patient, then causes the catheter to enter a blood vessel, and further removes the inner needle from the catheter to indwell the catheter.

During use, this type of catheter assembly is likely to deflect when a user brings a portion of the multi-structure needle exposed from a grip into contact with the treatment target to obliquely puncture the patient with the multi-structure needle. When the multi-structure needle is deflected in this manner, it becomes difficult to puncture the multi-structure needle. Therefore, the catheter assembly disclosed in US 2016/0256667 A has a structure for supporting the catheter by arranging two support portions to which the inner needle is fixed at positions near the outer periphery of the catheter at a distal end of the grip.

### SUMMARY

In the catheter assembly disclosed in US 2016/0256667 A, however, an upper portion and a lower portion supporting the outer side of the catheter are not directly held with each other, and thus, the upper portion and the lower portion are easily separated from each other, and there is a problem that the support performance of the multi-structure needle at the time of puncture is low.

Embodiments of the present invention have been developed to solve the above problem, and an object thereof is to provide a catheter assembly capable of more reliably supporting a catheter and favorably puncturing the catheter and an inner needle.

According to one aspect of the present invention, a catheter assembly includes: a catheter; a catheter hub fixed to the catheter; an inner needle inserted through the catheter; a grip that fixes and holds the inner needle; a first support portion configured as a member separate from the grip and capable of supporting an outer side of the catheter; and a second support portion that is configured as a member separate from the grip, has at least a part being located at a position facing the first support portion, and is capable of supporting the outer side of the catheter, the first support portion and the second support portion being directly held to be releasable from each other.

According to certain embodiments, it is possible to provide a catheter assembly capable of more reliably supporting the catheter and favorably puncturing the catheter and the inner needle.

### BRIEF DESCRIPTION OF DRAWINGS

FIG. 1 is a perspective view of a catheter assembly according to a first embodiment of the present invention;

FIG. 2 is an exploded perspective view of the catheter assembly of FIG. 1;

FIG. 3A is a perspective view of a catheter operation member as viewed from obliquely below; FIG. 3B is an enlarged perspective view illustrating a distal portion of the catheter operation member;

FIG. 4A is a perspective view illustrating a lower support member; FIG. 4B is a perspective view illustrating a state in which the lower support member is incorporated in a lower grip;

FIG. 5 is a partial front sectional view illustrating a distal portion of the catheter assembly;

FIG. 6 is a side view illustrating the distal portion of the catheter assembly;

FIG. 7A is a side view illustrating a state of the catheter assembly at the time of puncture; FIG. 7B is a side view illustrating a state of a conventional catheter assembly at the time of puncture;

FIG. 8 is a side view illustrating a state in which the catheter operation member is advanced after puncture;

FIG. 9 is a perspective view of a catheter assembly according to a second embodiment of the present invention; and

FIGS. 10A to 10C are plan views schematically illustrating operations of the catheter assembly according to the second embodiment.

### DETAILED DESCRIPTION

Hereinafter, preferred embodiments of the present invention will be described in detail with reference to the accompanying drawings.

### First Embodiment

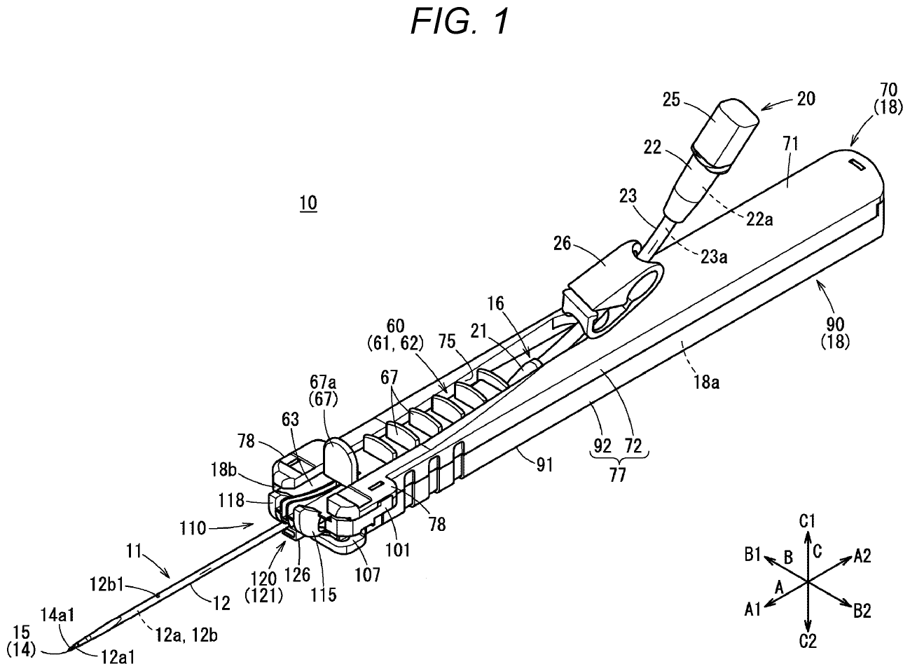

As illustrated in FIG. 1, a catheter assembly 10 according to a first embodiment of the present invention is a medical device used at the time of performing infusion, blood transfusion, blood sampling, or the like on a treatment target (patient), and a catheter 12 is inserted into and indwelled inside the body of the treatment target to cause the inside and outside of the body to communicate. This catheter assembly 10 enables insertion of a catheter 12 having a longer length (for example, a central venous catheter, a PICC, a mid-line catheter, and the like) than a peripheral venous catheter. Incidentally, the catheter assembly 10 may also be configured to enable insertion of the peripheral venous catheter. In addition, the catheter assembly 10 is not limited to the venous catheter, and may be configured to enable insertion of an arterial catheter such as a peripheral arterial catheter.

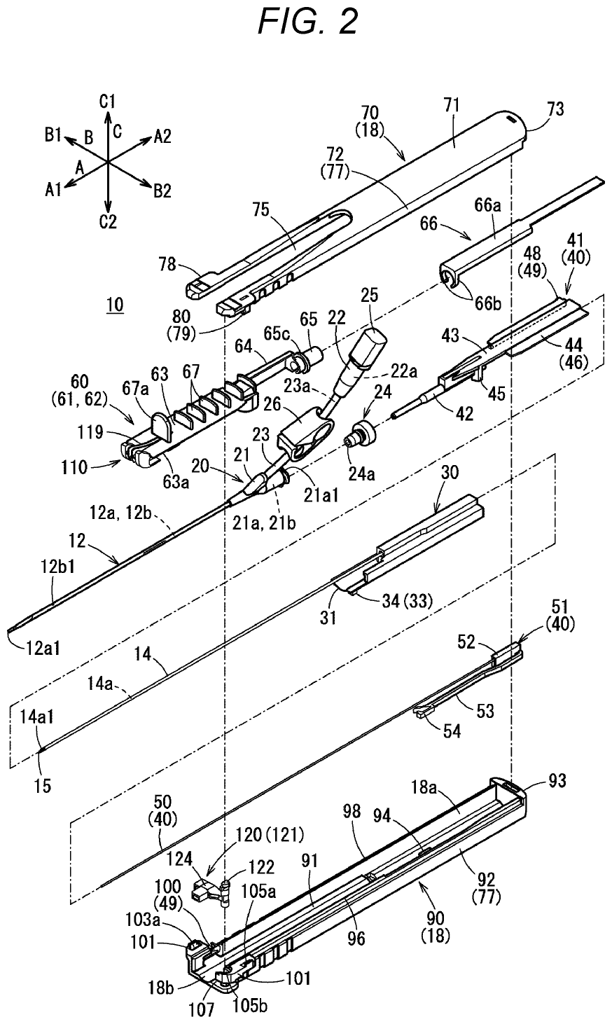

As illustrated in FIGS. 1 and 2, the catheter assembly 10 includes an inner and outer needle assembly 16 in which the catheter 12, an inner needle 14, a catheter hub 20, an inner needle hub 30, a safety member 40, and a catheter operation member 60 are assembled in a state before use (before puncture). Further, the catheter assembly 10 includes a grip 18 (housing) configured to accommodate the inner and outer needle assembly 16 and be gripped by a user.

In the inner and outer needle assembly 16 in the state before puncture, the inner needle 14 penetrates through the catheter 12 and the catheter hub 20, and a needle tip 15 of the inner needle 14 forms a multi-structure needle 11 protruding from a distal end of the catheter 12. The safety member 40 through which the inner needle 14 is inserted is arranged closer to a proximal side than the catheter hub 20, and the inner needle hub 30 holding the inner needle 14 is arranged on the proximal side of the safety member 40. The catheter operation member 60 is arranged above the catheter 12, the catheter hub 20, and the safety member 40, and causes advancement and retraction of these members under the operation of the user. The inner and outer needle assembly 16 including a portion of the multi-structure needle 11 on the proximal side is accommodated in the grip 18, and the inner needle hub 30 is fixed to the grip 18.

The catheter 12 according to the present embodiment is a tubular body having appropriate flexibility, and is configured as a multi-lumen type having a plurality of lumens 12a and 12b therein. The lumens 12a and 12b extend in the axial direction (arrow A direction) of the catheter 12, and communicate with a distal opening 12a1 and a distal opening 12b1, respectively, at the distal end of the catheter 12. For example, the lumen 12a is formed in a circular shape capable of accommodating the inner needle 14, and the lumen 12b is formed in an arc-shaped ellipse above the lumen 12a. A length of the catheter 12 is set to about 14 to 500 mm, preferably within a range of 30 to 400 mm, and more preferably within a range of 76 to 200 mm.

A constituent material of the catheter 12 is a soft resin material is suitable, and examples thereof include a fluorine-based resin such as polytetrafluoroethylene (PTFE), ethylene-tetrafluoroethylene copolymer (ETFE) and perfluoroalkoxy fluorine resin (PFA), an olefin-based resin such as polyethylene and polypropylene or a mixture thereof, polyurethane, polyester, polyamide, polyether nylon resin, a mixture of the olefin-based resin and ethylene-vinyl acetate copolymer, and the like. Note that it is a matter of course that the catheter 12 may be a single lumen type including only the lumen 12a through which the inner needle 14 is inserted without being limited to the multi-lumen type.

A proximal end of the catheter 12 is fixed to a distal end in the catheter hub 20 by an appropriate fixing means such as caulking, fusion, and adhesion. The catheter hub 20 is exposed on a skin of a treatment target in a state in which the catheter 12 has been inserted into a blood vessel of the treatment target, and indwells together with the catheter 12 by being pasted with a tape or the like.

The catheter hub 20 has two separate hubs (main hub 21 and sub hub 22) corresponding to the multi-lumen type catheter 12. The main hub 21 is a member directly connected to the catheter 12, and the sub hub 22 is a member connected to the main hub 21 through a tube 23.

A constituent material of the catheter hub 20 (main hub 21 and sub hub 22) is not particularly limited, but a thermoplastic resin such as polypropylene, polycarbonate, polyamide, polysulfone, polyarylate, and a methacrylate-butylene-styrene copolymer may be preferably applied, for example.

The main hub 21 is a tubular body extending in parallel with the axis on the proximal side of the catheter 12, and the tube 23 is connected to a predetermined position on an outer peripheral surface of the main hub 21. An internal space 21a communicating with the lumen 12a and an internal space 21b communicating with the lumen 12b are provided inside the main hub 21. A proximal end of the internal space 21a communicates with a proximal opening 21a1 of the main hub 21. On the other hand, the internal space 21b is separated from the internal space 21a and communicates with a lumen 23a of the tube 23 inserted into and fixed to the main hub 21.

In the state before puncture, a valve member 24 is inserted into the main hub 21 from the proximal opening 21a1 toward the back side (arrow A1 side) of the internal space 21a. A valve hole 24a that can be elastically opened and closed is provided at the axial center of the valve member 24. Distal ends of the inner needle 14 and the safety member 40 are inserted into the valve hole 24a in the state before puncture of the valve member 24, so that an inner surface of the valve hole 24a and an outer surface of the safety member 40 are in close contact with each other. As a result, the valve member 24 brings the catheter hub 20 and the safety member 40 into a fitted state, and inhibits leakage of blood from the proximal opening 21a1 of the main hub 21 at the time of puncture with the inner needle 14.

In addition, the sub hub 22 is formed in a tubular shape having the same thickness as the main hub 21, and a proximal end of the tube 23 is inserted from a distal end of the sub hub 22 and fixed thereto. An internal space 22a communicating with the lumen 23a of the tube 23 is formed inside the sub hub 22. A proximal end of the internal space 22a communicates with a proximal opening (not illustrated) of the sub hub 22. In the state before puncture, a closing member 25 that closes the proximal opening is connected to the sub hub 22.

The tube 23 is configured to have flexibility similarly to the catheter 12. A clamp 26 capable of opening and closing the lumen 23a of the tube 23 is attached in advance to an intermediate position in the extending direction of the tube 23.

Meanwhile, the inner needle 14 of the catheter assembly 10 is configured as a hollow tubular body having rigidity capable of puncturing a skin of a living body. The needle tip 15, which is sharp, is provided at the distal end of the inner needle 14. A hollow portion 14a is formed inside the inner needle 14 to penetrate along the arrow A direction, and the hollow portion 14a communicates with a distal opening 14a1 provided in the needle tip 15.

Examples of a constituent material of the inner needle 14 include a metal material such as stainless steel, aluminum or an aluminum alloy, and titanium or a titanium alloy, a hard resin, ceramics, and the like. The inner needle 14 is firmly fixed to the inner needle hub 30 by an appropriate fixing means such as fusion, adhesion, and insert molding.

The inner needle hub 30 directly holds the inner needle 14 and is fixed to the grip 18 through a grip fixing portion 31 (lower wall) formed on an arrow C2 side. The grip fixing portion 31 has a lower surface provided with a plurality of fixing protrusions 34 that protrude short in the downward direction and constitute an attachment mechanism 33 with the grip 18.

The safety member 40 is configured to be inserted and fitted into the main hub 21 and the valve member 24 of the catheter hub 20 to follow the catheter hub 20 that is moving. The safety member 40 includes a cover body 41 that covers the outer side of the inner needle 14 along with advancement, a blunt needle 50 protruding from the needle tip 15 of the inner needle 14 after puncture, and the blunt needle hub 51 holding the blunt needle 50.

The cover body 41 includes: a distal cover portion 42 that has a cylindrical shape and accommodates and protects the inner needle 14 after puncture; a proximal extending portion 43 extending from an upper portion of the distal cover portion 42 to the proximal side (arrow A2 side); and a pair of protruding pieces 44 protruding outward in the width direction from the proximal extending portion 43. In addition, an engagement projection 45 with which the blunt needle hub 51 is engaged is provided at a point where the proximal extending portion 43 and the distal cover portion 42 are connected.

The distal cover portion 42 is inserted into and brought into close contact with the valve member 24 on the distal side, thereby being frictionally fitted to the catheter hub 20 including the valve member 24. In the state before puncture, a proximal end of the distal cover portion 42 faces a distal end of the inner needle hub 30. The proximal extending portion 43 connected to the distal cover portion 42 extends to a proximal end in the grip 18 along an upper portion of the inner needle hub 30 in the state before puncture.

The pair of protruding pieces 44 protrudes outward in the width direction (arrow B direction) from the inner needle hub 30 and extends to the vicinity of a side wall 77 of the grip 18 (above rail walls 96 and 98 to be described later). The respective protruding pieces 44 constitute a guide mechanism 46 that performs guide in the arrow A direction in cooperation with the grip 18 when the safety member 40 moves. In addition, a locked protrusion 48 is provided on a side of the protruding piece 44 on an arrow B1 side. The locked protrusion 48 is locked to a locking portion 100 of the grip 18 at the advanced position at which the safety member 40 has advanced, thereby constituting one of safety movement restricting mechanisms 49 that restrict the advancement and retraction of the cover body 41.

The blunt needle 50 of the safety member 40 is a rod member (round rod) configured to inhibit erroneous puncture of the catheter 12 and the inner needle 14 with respect to a living body, and is movably accommodated in the hollow portion 14a of the inner needle 14. A distal end of the blunt needle 50 is formed in a shape blunter than the needle tip 15 of the inner needle 14 (for example, to have a polished flat surface), and is arranged at a position near a proximal end of the distal opening 14a1 in the hollow portion 14a of the inner needle 14 in the state before puncture. The distal end of the blunt needle 50 is exposed from the needle tip 15 (distal opening 14a1) as the safety member 40 advances.

The blunt needle hub 51 is configured to be relatively movable with respect to the inner needle 14, the inner needle hub 30, and the grip 18 by holding the blunt needle 50 and engaging with the engagement projection 45 of the cover body 41. The blunt needle hub 51 includes a blunt needle holding portion 52 that holds the blunt needle 50 on the arrow A2 side, and an arm portion 53 extending from the blunt needle holding portion 52 to the arrow A1 side.

The blunt needle holding portion 52 is arranged in a space on the proximal side of a point where the inner needle 14 is fixed in the inner needle hub 30. When a distal end surface of the blunt needle holding portion 52 comes into contact with the fixing point of the inner needle 14 as the blunt needle hub 51 advances, the subsequent advancement of the blunt needle hub 51 is inhibited.

The entire extending portion of the arm portion 53 is configured to be elastically deformable in the width direction, and an engagement end 54 to be engaged with the engagement projection 45 in the state before puncture is provided at the distal end of the arm portion 53. When the cover body 41 further advances at a stage where the movement of the blunt needle hub 51 is restricted, the engagement end 54 is elastically deformed as appropriate to release the engagement with the engagement projection 45.

Note that the safety member 40 is not limited to the above configuration as long as erroneous puncture of the needle tip 15 of the inner needle 14 can be inhibited. For example, the safety member 40 may include only the cover body 41 without including the blunt needle 50 and the blunt needle hub 51.

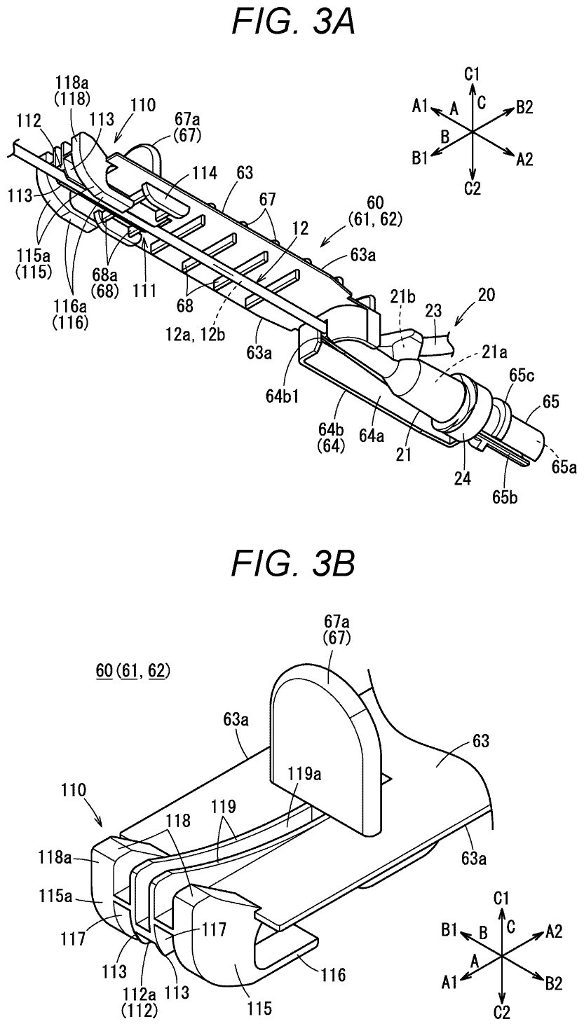

As illustrated in FIGS. 2, 3A, and 3B, the catheter operation member 60 constitutes an operation unit 61 to be operated by the user in the catheter assembly 10. In addition, the catheter operation member 60 according to the present embodiment is also a first support portion 62 that supports the outer side of the catheter 12 (multi-structure needle 11) at the time of puncture with the multi-structure needle 11. A material forming the catheter operation member 60 is not particularly limited, and, for example, the materials exemplified for the catheter hub 20 can be appropriately selected.

Specifically, the catheter operation member 60 includes: an operation plate portion 63 (extending portion) extending in the arrow A direction; a hub engagement portion 64 connected to a proximal end of the operation plate portion 63 and engaged with the catheter hub 20; and an operation member tubular portion 65 that is connected to a proximal end of the hub engagement portion 64 and accommodates the safety member 40. In addition, the catheter operation member 60 includes a cover member 66 which extends in the proximal direction from the hub engagement portion 64 and covers the safety member 40.

The operation plate portion 63 is a site on which a finger of the user abuts and that slides by advancing/retracting operation in the extending direction of the catheter 12. The operation plate portion 63 is formed to be thin, and thus, has flexibility capable of being curved in a direction away from the multi-structure needle 11 (direction in which the operation unit 61 and a second support portion 121 to be described later overlap each other). The operation plate portion 63 has side edges 63a, which extend in the arrow A direction, on both sides in the width direction. A plurality of tabs 67 are provided on an upper surface of the operation plate portion 63. A tab 67a at the most distal end among the plurality of tabs 67 protrudes more than the other tabs 67. Further, a plurality of ribs 68 protrude short from a lower surface of the operation plate portion 63. The catheter 12 is arranged below the plurality of ribs 68.

An operation support portion 110 configured to support the catheter 12 is provided in a distal region of the operation plate portion 63. The operation support portion 110 is formed on the lower surface side of the operation plate portion 63, and has a catheter support structure 111 that arranges the catheter 12 (multi-structure needle 11) inward in the width direction.

The catheter support structure 111 is provided at the central portion in the width direction of the catheter operation member 60 and extends from a distal end toward the arrow A2 side by a predetermined length. A proximal end of the catheter support structure 111 reaches the arrow A2 side farther than the tab 67a. The catheter support structure 111 includes: a base portion 112 that can come into contact with an arrow C1 side of the catheter 12; and a pair of ridge portions 113 that can come into contact with the catheter 12 in the arrow B direction.

The base portion 112 is formed to be slightly shifted to the lower side (arrow C2 side) from the operation plate portion 63, and a vertical position of a lower surface 112a facing the catheter 12 substantially coincides with a protruding end of the rib 68.

The pair of ridge portions 113 is configured to sandwich the base portion 112 therebetween, and protrudes from the lower surface of the operation plate portion 63 to the arrow C2 side. A protruding amount of each of the ridge portions 113 from the operation plate portion 63 is larger than a protruding amount of the base portion 112 from the operation plate portion 63. For example, each of the ridge portions 113 protrudes further from the base portion 112 by a length substantially equal to an outer diameter of the catheter 12.

Some ribs 68a on the distal side among the plurality of ribs 68 provided on the lower surface of the operation plate portion 63 are provided to be continuous with the proximal side of the pair of ridge portions 113. These ribs 68a are continuous with side ribs 114 extending short in the arrow A direction on the outer side in the width direction. The side rib 114 protrudes to the arrow C2 side more than the rib 68a, and has a function of guiding the advancement and retraction of the catheter operation member 60 with respect to the grip 18.

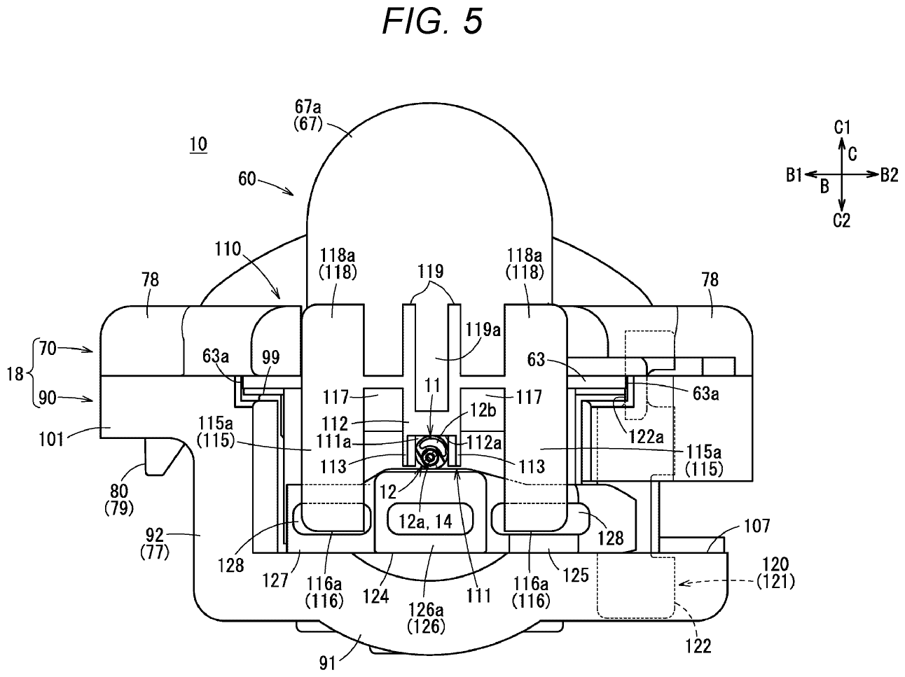

In the state before puncture, the catheter support structure 111 arranges the catheter 12 in a support space 111a (see FIG. 5) surrounded by the base portion 112 and the pair of ridge portions 113. Since the catheter support structure 111 is long in the arrow A direction, the catheter 12 is favorably maintained in a linearly extending state in a long range of a distal region of the catheter operation member 60.

In addition, the operation support portion 110 includes, on the lower surface side of the operation plate portion 63 and on the outer side of the catheter support structure ill in the width direction, a pair of lower protruding blocks 115 (protruding portions) protruding to the arrow C2 side, and holding portions 116 respectively extending from protruding ends of the lower protruding blocks 115. The pair of holding portions 116 is held by a lower support member 120 (second support portion 121), which will be described later, in the state before puncture to inhibit the distal end of the catheter operation member 60 from being separated in the arrow C direction (direction in which the operation unit 61 and the second support portion 121 overlap each other).

That is, the pair of lower protruding blocks 115 and the pair of holding portions 116 have a shape folded back from the distal end of the operation plate portion 63. In addition, each of the lower protruding blocks 115 and each of the holding portions 116 are provided with a predetermined gap 117 with respect to the catheter support structure 111.

The pair of lower protruding blocks 115 protrudes to the arrow C2 side more than the catheter support structure 111 (the pair of ridge portions 113). In addition, a width (thickness) of each of the lower protruding blocks 115 is sufficiently larger than a width (thickness) of the ridge portion 113. A distal end surface 115a of each of the lower protruding blocks 115 is formed to be curved to the arrow A2 side as proceeding to the arrow C2 side.

The pair of holding portions 116 is continuous with lower ends (the protruding ends on the arrow C2 side) of the lower protruding blocks 115, respectively, and linearly extends toward the arrow A2 side. An extending length of each of the holding portions 116 may be appropriately designed according to a position of the lower support member 120, and is formed to be longer than a length of the lower protruding block 115 in the arrow A direction, for example. In addition, each of the holding portions 116 is formed to have the same width as each of the lower protruding blocks 115. A thickness of the holding portion 116 in the vertical direction is smaller than the width of the holding portion 116.

A lower surface 116a of the holding portion 116 is smoothly continuous with the distal end surface 115a of the lower protruding block 115. As a result, when the catheter operation member 60 comes into contact with the body surface of the treatment target at a stage where the catheter operation member is delivered from the grip 18 to some extent, the distal end surface 115a and the lower surface 116a guide the catheter operation member 60 to be separated from an insertion point of the catheter 12.

Further, the operation support portion 110 has a pair of upper protruding blocks 118, which protrude short toward the arrow C1 side at the same position as the lower protruding block 115 in the width direction on the upper surface side of the operation plate portion 63. A width of each of the upper protruding blocks 118 coincides with the width of each of the lower protruding blocks 115. A distal end surface 118a of each of the upper protruding blocks 118 is flush with an upper end of the distal end surface 115a of the lower protruding block 115.

Further, a pair of reinforcing pieces 119 (reinforcing portions) that inhibits deflection in a direction away from the catheter 12 is provided on the inner side in the width direction of the pair of upper protruding blocks 118. Each of the reinforcing pieces 119 is provided at the same position as each of the ridge portions 113 in the width direction and protrudes to the opposite side (arrow C1 side) of each of the ridge portions 113. Each of the reinforcing pieces 119 protrudes from the upper surface of the operation plate portion 63 to the same extent as the upper protruding block 118 at the distal end, is formed to be gradually lowered while being curved from the distal end to the arrow A2 side, and is in contact with the tab 67a. Since the operation plate portion 63 does not exist between the respective reinforcing pieces 119, a groove portion 119a in which an upper surface of the base portion 112 of the catheter support structure 111 is exposed is formed.

Meanwhile, the hub engagement portion 64 continuous with the proximal end of the operation plate portion 63 has an accommodation chamber 64a for accommodating the main hub 21, and has a wall portion 64b on the arrow B1 side to be formed in a shape in which the arrow B2 side is cut out (shape in which the accommodation chamber 64a is opened). This shape is formed to expose the sub hub 22 and the tube 23 of the catheter hub 20 configured as the multi-lumen type. The distal side of the hub engagement portion 64 extends such that the wall portion 64b on the arrow B1 side wraps around the accommodation chamber 64a. The wall portion 64b on the distal side is provided with a gap 64b1 that is narrower than a diameter of the catheter hub 20 and from which only the catheter 12 (multi-structure needle 11) extends.

The operation member tubular portion 65 is formed in a cylindrical shape that protrudes short in the proximal direction from a proximal end surface of the hub engagement portion 64. A communicating space 65a that communicates with the accommodation chamber 64a and in which the safety member 40 (cover body 41) is arranged is provided on the inner side of the operation member tubular portion 65. In addition, a slit 65b communicating with the accommodation chamber 64a and the communicating space 65a is formed in the lower portion of the operation member tubular portion 65. Further, an arcuate rib 65c that protrudes in the circumferential direction and restricts the movement of the cover member 66 is provided on the outer peripheral surface of the operation member tubular portion 65.

Returning to FIG. 2, the cover member 66 is mounted on the catheter operation member 60 to inhibit the user from directly coming into contact with the safety member 40. The cover member 66 includes a body portion 66a that covers the upper side (side on which a hand is located when being gripped by the user) of the safety member 40, and a pair of attachment leg portions 66b provided at a distal end of the body portion 66a and attached to the operation member tubular portion 65. The pair of attachment leg portions 66b is engaged between the proximal end surface of the hub engagement portion 64 and the arcuate rib 65c.

In addition, the grip 18 of the catheter assembly 10 is formed to have an appropriate thickness that can be easily held by the user, and extends along the arrow A direction. The accommodation space 18a in which the catheter 12, the catheter hub 20, the safety member 40, and the catheter operation member 60 can advance and retract is formed in the grip 18. The accommodation space 18a communicates with a distal opening portion 18b of the grip 18. The grip 18 is configured by assembling an upper grip 70 and a lower grip 90, which can be divided in the arrow C direction, with each other.

The upper grip 70 has a ceiling wall 71, a pair of upper side walls 72, and an upper rear wall 73, and is formed in a recessed shape (bowl shape) that is opened downward. The pair of upper side walls 72 constitutes side walls 77 on both sides in the width direction of the grip 90 together with a lower side wall 92 of the lower grip 18.

In addition, the ceiling wall 71 has an operation member exposure notch 75 at the center in the arrow B direction closer to the distal side than an intermediate portion in the arrow A direction. The operation member exposure notch 75 is opened at the distal end to communicate with the accommodation space 18a, and exposes the tube 23 of the catheter hub 20 and the tab 67 of the catheter operation member 60 so as to be capable of advancing and retracting. Further, the upper grip 70 has a pair of upper projecting pieces 78 at the distal end, and a fixing hook 80 constituting a fixing mechanism 79 between the upper grip 70 and the lower grip 90 is provided on lower surfaces of the respective upper projecting pieces 78 and the upper rear wall 73.

The lower grip 90 has a bottom wall 91, a pair of lower side walls 92, and a lower rear wall 93, and is formed in a recessed shape (bowl shape) opened upward. A predetermined range on the arrow A2 side of the bottom wall 91 is a mounting target portion on which the inner needle hub 30 is mounted, and a plurality of (three in the present embodiment) mounting holes 94 (a part of the attachment mechanism 33) into which the fixing protrusion 34 of the inner needle hub 30 can be fitted are provided.

In addition, the pair of lower side walls 92 has the rail walls 96 and 98 in the upper portion thereof, and the upper side wall 72 of the upper grip 70 is arranged on the outer side of the rail walls 96 and 98 in the width direction in an assembled state. In the assembled state, the pair of side edges 63a of the catheter operation member 60 and the protruding pieces 44 of the safety member 40 are slidably arranged on the pair of rail walls 96 and 98. In addition, the locked protrusion 48 of the cover body 41 is arranged between the upper side wall 72 on the arrow B1 side and the rail wall 98.

The lower side wall 92 on the arrow B1 side is provided with a locking portion 100 that is engaged with the locked protrusion 48 to define an advancement limit of the cover body 41 and restrict the retraction of the cover body 41 when the cover body 41 of the safety member 40 advances. That is, the locking portion 100 constitutes the safety movement restricting mechanism 49 together with the locked protrusion 48. The catheter assembly 10 can favorably cover (protect) the inner needle 14 after puncture with the cover body 41 by restricting detachment of the safety member 40 from the grip 18.

A pair of projecting bodies 101 protruding outward in the width direction from the pair of lower side walls 92 is provided at the distal end of the lower grip 90. The projecting body 101 on the arrow B1 side has a first fixing hole 103a that is a part of the fixing mechanism 79. The projecting body 101 on the arrow B2 side has a second fixing hole 105a that is a part of the fixing mechanism 79. In addition, the projecting body 101 on the arrow B2 side has a movement space 107 in which the lower support member 120 is rotatably movable.

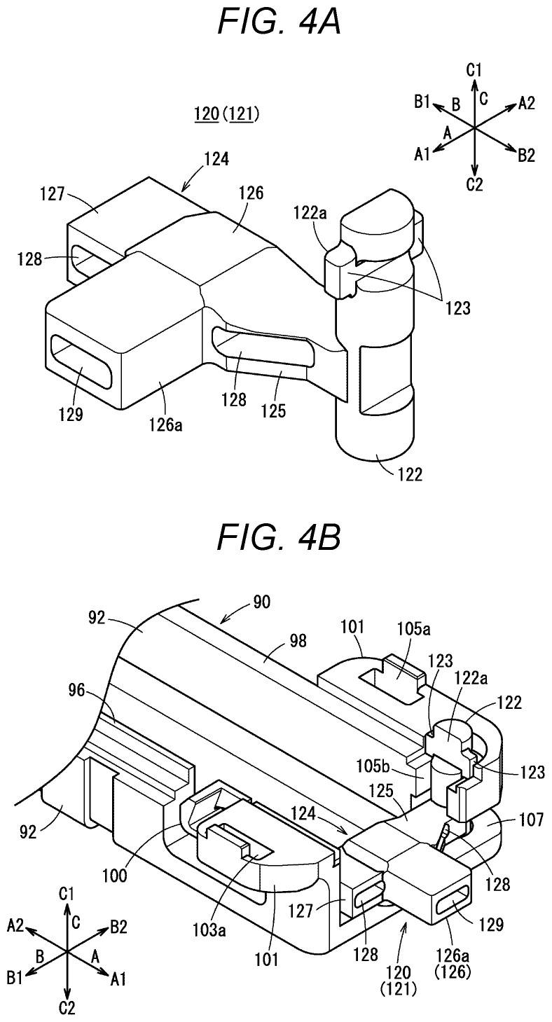

As illustrated in FIGS. 4A and 4B, the lower support member 120 is rotatably attached to the grip 18, and constitutes the second support portion 121 that supports the catheter 12 (multi-structure needle 11) extending on the lower side of the catheter operation member 60 in the state before puncture. In addition, the lower support member 120 rotates by contact with the wall portion 64b of the hub engagement portion 64 when the catheter operation member 60 advances, and enables the catheter operation member 60 (and the catheter hub 20 and the safety member 40) to be delivered from the accommodation space 18a.

The lower support member 120 includes a shaft 122 extending in the arrow C direction and a supporting body portion 124 protruding in a direction orthogonal to the axial direction of the shaft 122. The shaft 122 has an upper portion provided with a guide plane 122a continuous with an upper end of the rail wall 96 and a pair of small projections 123 having the guide plane 122a. The side edge 63a of the catheter operation member 60 is close to the guide plane 122a in the state before puncture. As a result, the rotation of the lower support member 120 is restricted.

The supporting body portion 124 has an appropriate thickness in the arrow C direction, and is connected to the lower side (arrow C2 side) of an intermediate position of the shaft 122 in the arrow C direction. The supporting body portion 124 is formed to be thicker than a thickness of the bottom wall 91 of the lower grip 90, for example. The supporting body portion 124 is formed by providing a coupling portion 125 connected to the shaft 122, a central portion 126 located at the central portion in the width direction, and a protruding end 127 located at a position farthest from the shaft 122 to be continuous with each other from the arrow B2 side to the arrow B1 side.

The coupling portion 125 is formed to have the same width as an outer diameter of the shaft 122 on the arrow B2 side in a plan view, and is formed in a substantially triangular shape that is gradually inclined toward the arrow A1 side as being separated from the shaft 122.

The central portion 126 slightly protrudes to the arrow C1 side from the coupling portion 125 and the protruding end 127, and basically has a configuration in which the central portion 126 supports the lower side of the catheter 12. In addition, the central portion 126 has a protrusion 126a that protrudes to the arrow A1 side (distal side) more than a distal end of the coupling portion 125. The protrusion 126a protrudes in the distal direction from the distal end of the grip 18 in a state in which the lower support member 120 is assembled to the grip 18. A protruding amount (length in the arrow A direction) of the protrusion 126a is not particularly limited, but for example, may be set to be longer than (or substantially equal to) a length of the protruding end 127 in the arrow A direction.

The protruding end 127 is formed in a rectangular shape in a plan view, and is arranged at a position near the lower side wall 92 on the arrow B2 side of the grip 18 (lower grip 90). A thickness of the protruding end 127 in the arrow C direction substantially coincides with a thickness of the coupling portion 125 in the arrow C direction.

The supporting body portion 124 has insertion target portions 128 into which the pair of holding portions 116 of the catheter operation member 60 is inserted, at the coupling portion 125 and the protruding end 127, respectively. The pair of insertion target portions 128 extends along the arrow A direction of the supporting body portion 124 and penetrates a distal end surface and a proximal end surface of the supporting body portion 124. That is, each of the holding portions 116 and each of the insertion target portions 128 extend along the extending direction of the catheter 12.

A length of each of the insertion target portions 128 in the arrow C direction substantially coincides with a length of each of the holding portions 116 in the arrow C direction. Inner surfaces of the supporting body portion 124 respectively constituting the insertion target portions 128 come into contact with each other with an appropriate frictional force that enables each of the holding portions 116 to slide in the distal direction in a state in which each of the holding portions 116 is inserted. Note that the supporting body portion 124 also has a lightening hole 129 penetrating in the arrow A direction in the central portion 126 in order to improve the molding accuracy of the supporting body portion 124.

The lower support member 120 configured as described above is inserted along the bearing notch 105b from above the lower grip 90 in a posture in which the supporting body portion 124 faces the arrow B1 side. At this time, the lower support member 120 is smoothly inserted into a bearing notch 105b as the vicinity of the coupling portion of the supporting body portion 124 passes through an opening portion of the bearing notch 105b on the accommodation space 18a side. When the upper grip 70 and the lower grip 90 are mounted, the upper end of the shaft 122 supported by the lower grip 90 is pivotally supported by the upper grip 70.

In the state before puncture of the lower support member 120, the side edge 63a of the catheter operation member 60 is present on the guide plane 122a, and thus, the supporting body portion 124 is restricted from rotating and stands by to be capable of supporting the catheter 12. As a result, the supporting body portion 124 supports the catheter 12 from below to suppress the deflection of the catheter 12. When the catheter operation member 60 advances from the grip 18, the lower support member 120 becomes rotatable as the side edge 63a comes out of the guide plane 122a, and allows the catheter hub 20, the catheter operation member 60, and the safety member 40 to be delivered.

In the catheter assembly 10 configured as described above, the inner and outer needle assembly 16 (catheter 12, inner needle 14, catheter hub 20, inner needle hub 30, safety member 40, and catheter operation member 60) is assembled first at the time of assembly. Thereafter, the inner and outer needle assembly 16 is arranged on the lower grip 90 incorporating the lower support member 120, and the upper grip 70 is mounted on the inner and outer needle assembly 16 and the lower grip 90.

When the inner and outer needle assembly 16 is assembled to the lower grip 90, the pair of holding portions 116 of the catheter operation member 60 is inserted into the pair of insertion target portions 128 of the lower support member 120. That is, the inner and outer needle assembly 16 is lowered toward an opening portion on the arrow C1 side of the lower grip 90 in a state in which the inner and outer needle assembly 16 is slightly shifted to the distal side with respect to the lower grip 90. Then, the pair of holding portions 116 is inserted from distal ends of the pair of insertion target portions 128 by moving the inner and outer needle assembly 16 to the arrow A2 side in a state in which the pair of side edges 63a of the catheter operation member 60 is in contact with the rail walls 96 and 98. As a result, the catheter operation member 60 and the lower support member 120 can be easily held and engaged with each other.

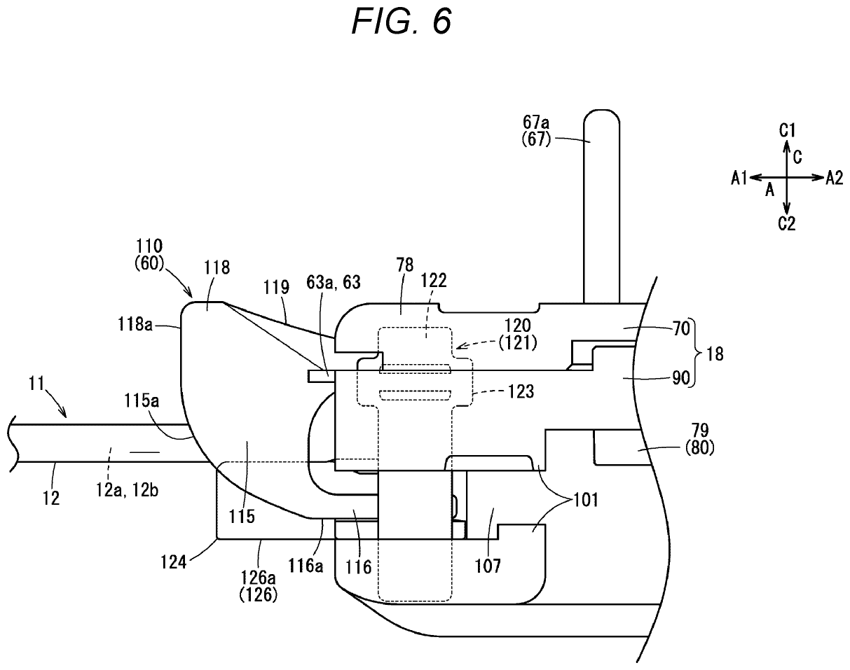

As illustrated in FIGS. 5 and 6, in the distal portion of the catheter assembly 10, the catheter 12 is arranged (in the support space 111a) between the catheter operation member 60 and the lower support member 120 engaged with each other. At this time, the catheter operation member 60 and the lower support member 120 may come into contact with an outer peripheral surface of the catheter 12, or may have a slight clearance with respect to the catheter 12. Even when the clearance is provided, the catheter 12 moves slightly and is supported by the catheter operation member 60 and the lower support member 120 at the time of puncture.

In addition, the catheter operation member 60 is inhibited from moving in the arrow C direction (vertical direction) by the lower support member 120 in a state in which the pair of holding portions 116 is inserted into the pair of insertion target portions 128. On the other hand, each of the holding portions 116 is slidable to the arrow A1 side with respect to the lower support member 120, and thus, can easily advance the catheter operation member 60.

Further, in the catheter operation member 60, the pair of side edges 63a is arranged in a guide space 99 (space sandwiched between the upper side wall 72 of the upper grip 70 and the pair of rail walls 96 and 98 of the lower grip 90) of the guide mechanism 46 of the grip 18. Therefore, the catheter operation member 60 engages with the pair of side edges 63a not to be detachable in the arrow C direction (direction in which the catheter operation member 60 and the lower support member 120 overlap each other), and the deflection in the grip 18 is more reliably restricted. In addition, the rotation of the lower support member 120 is restricted as the side edge 63a on the arrow B2 side of the catheter operation member 60 is close to the guide plane 122a. Therefore, even when each of the holding portions 116 slides with respect to each of the insertion target portions 128, the lower support member 120 can continue to stand by at the position in the state before puncture (the position to support the catheter 12 from below).

The catheter assembly 10 according to the present embodiment is basically configured as described above, and operations thereof will be described hereinafter.

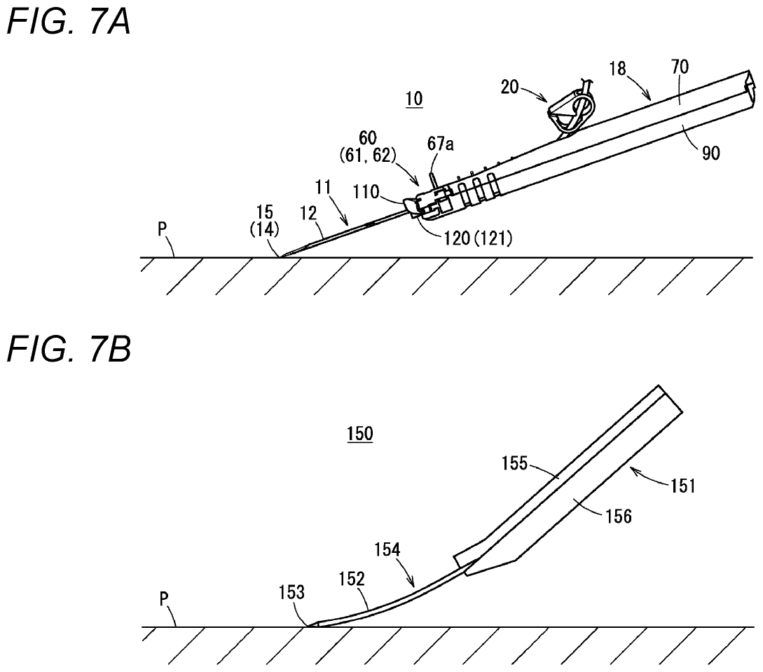

As described above, the catheter assembly 10 is used when infusion, blood transfusion, blood sampling, or the like is performed on the treatment target (living body). As illustrated in FIG. 7A, the user grips and operates the grip 18 to puncture a treatment target P with the multi-structure needle 11 at the time of using the catheter assembly 10.

At the time of puncture, the catheter 12 (multi-structure needle 11) is sandwiched and supported by the catheter operation member 60 and the lower support member 120 at the distal end of the grip 18. In a supporting state, the catheter support structure 111 and the lower support member 120 have walls arranged in four directions (the arrow B direction and the arrow C direction) of the catheter 12, and restrict the movement of the catheter 12 at supporting points. In addition, deformation of the catheter operation member 60 in the arrow C direction (vertical direction) is inhibited as the pair of holding portions 116 is inserted into the pair of insertion target portions 128 of the lower support member 120.

Here, as illustrated in FIG. 7B, a conventional catheter assembly 150 has a configuration in which an upper grip 155 and a lower grip 156 supporting a catheter 152 and an inner needle 153 (multi-structure needle 154) are easily separated from each other at a distal end of a grip 151. Therefore, in the catheter assembly 150, when an upward force is applied from the multi-structure needle 154 in contact with the treatment target P at the time of puncture, the upper grip 155 is separated from the lower grip 156, and the multi-structure needle 154 is deflected.

Meanwhile, in the catheter assembly 10, the catheter operation member 60 directly supporting the outer side of the catheter 12 and the lower support member 120 are engaged with each other as illustrated in FIGS. 5 and 6. Therefore, in the catheter assembly 10, the catheter operation member 60 is not deformed upward even when an upward force is applied to the catheter operation member 60 from the multi-structure needle 11 in contact with the treatment target P at the time of puncture, and deflection of the multi-structure needle 11 is suppressed. As a result, the user can favorably implant the catheter 12 (multi-structure needle 11) into the body.

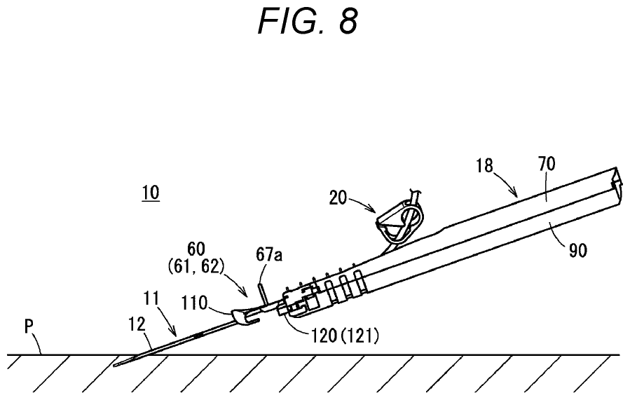

When the treatment target P is punctured with the multi-structure needle 11 and the needle tip 15 reaches a blood vessel, the user performs an advancing operation of the catheter operation member 60 to advance the catheter 12 beyond the inner needle 14 to be inserted into the blood vessel as illustrated in FIG. 8. The catheter operation member 60 relatively advances with respect to the lower support member 120 in accordance with the advancing operation of the user. Then, when each of the holding portions 116 comes out of each of the insertion target portions 128, the catheter operation member 60 can be curved upward (in a direction away from the catheter 12) on the distal side of the grip 18.

The lower support member 120 is restricted from rotating as the side edge 63a of the catheter operation member 60 is close thereto. Therefore, the holding portion 116 does not rotate even if coming out of the lower support member 120, and can favorably support the catheter 12. The lower support member 120 is rotatable when a proximal end of the side edge 63a of the catheter operation member 60 (operation plate portion 63) comes out of the grip 18. The lower support member 120 relatively rotates with respect to the grip 18 by coming into contact with and being pushed by the hub engagement portion 64 (wall portion 64b) of the catheter operation member 60 in the advancement process. As a result, members (the catheter hub 20 and the safety member 40) on the proximal side of the hub engagement portion 64 can favorably come out of the distal opening portion 18b.

The catheter 12 and the catheter hub 20 are removed from a distal end of the grip 18 by the advancing operation of the user, and then, the safety member 40 protrudes from the distal end of the grip 18. When the catheter operation member 60 is further advanced, the locked protrusion 48 of the safety member 40 moves to the locking portion 100 (an advanced position) of the grip 18. At this time, a distal end of the safety member 40 is exposed from the grip 18 and advances beyond a distal end of the inner needle 14 to cover the inner needle 14, thereby activating an erroneous puncture inhibition function.

At the advanced position, the safety member 40 is in a locked state in which movement in both a distal direction and a proximal direction is stopped without coming out of the grip 18. As a result, when the catheter 12, the catheter hub 20, and the catheter operation member 60 further advance, the safety member 40 is separated from these members. The engagement between the catheter operation member 60 and the catheter hub 20 can be released as the catheter operation member 60 and the safety member 40 are separated from each other in the catheter assembly 10. Therefore, the catheter 12 and the catheter hub 20 are separated from the lower side of the catheter operation member 60.

The catheter 12 and the catheter hub 20 are detached from the catheter operation member 60 and indwelled in the treatment target P. After the indwelling, a connector (not illustrated) of another medical device is connected to the catheter hub 20. On the other hand, the inner needle 14, the inner needle hub 30, the safety member 40, and the grip 18 in the state of being integrated are appropriately discarded by the user.

Incidentally, the present invention is not limited to the above-described embodiment, and various modifications can be made in accordance with a gist of the invention. For example, the catheter operation member 60 and the lower support member 120 are not limited to the engagement between the pair of holding portions 116 and the pair of insertion target portions 128, and may adopt a configuration in which one or three or more holding portions 116 and insertion target portions 128 are engaged with each other.

### Second Embodiment

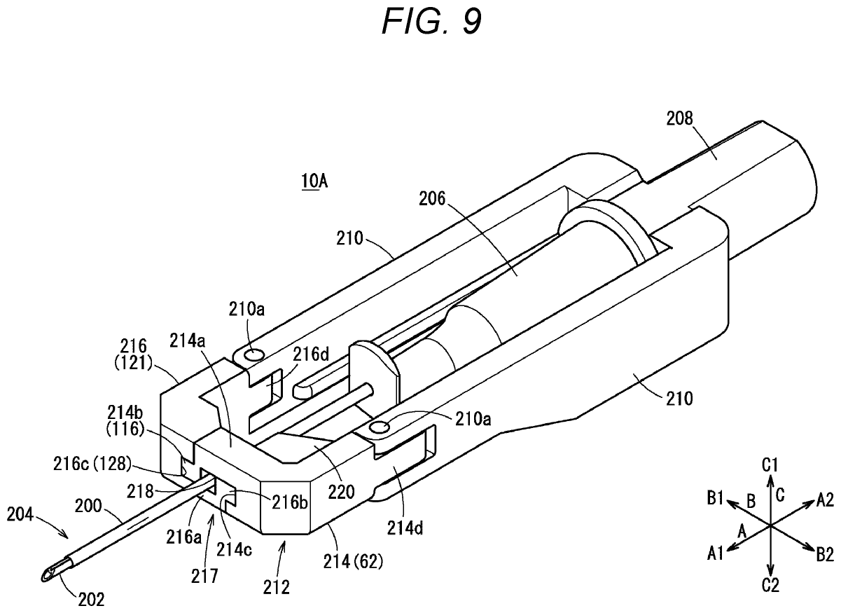

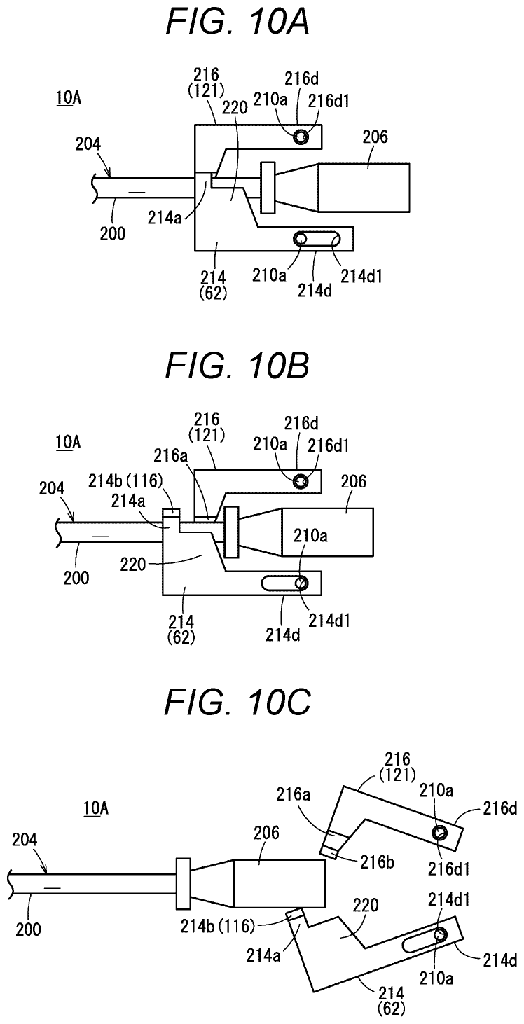

Next, a catheter assembly 10A according to a second embodiment of the present invention will be described with reference to FIGS. 9 and 10A to 10C. The catheter assembly 10A according to the second embodiment is different from the catheter assembly 10 according to the first embodiment in that a multi-structure needle 204 including a catheter 200 and an inner needle 202 is sandwiched and supported laterally.

Specifically, the catheter 200 is configured for a peripheral venous catheter, and is fixed and held to a catheter hub 206 on the proximal side (arrow A2 side). An inner needle hub 208 is provided on the arrow A2 side of the catheter hub 206, and a pair of grips 210 configured to be gripped by the user at the time of puncture is continuously provided on both sides of the inner needle hub 208 in the width direction (arrow B direction). The pair of grips 210 extends on the sides of the catheter hub 206 in the arrow A direction, and a support structure 212 that supports the outer side of the catheter 200 (multi-structure needle 204) at the time of puncture is provided on the distal side (arrow A1 side) thereof.

The support structure 212 includes a first support member 214 (first support portion 62) connected to the grip 210 on the arrow B2 side and a second support member 216 (second support portion 121) connected to the grip 210 on arrow B1 side. The first and second support members 214 and 216 further protrude from the grips 210 to the arrow A1 side in a plan view, and are formed in substantially L shapes bent in directions approaching each other at a predetermined position. In addition, the first and second support members 214 and 216 are rotatably connected to the grips 210 by shaft pins 210a provided at the distal ends of the grips 210, respectively.

The first and second support members 214 and 216 constitute a hook mechanism 217 that supports the catheter 200 by protruding ends on the inner side in the width direction and allows the protruding ends to be caught with each other. Specifically, the first support member 214 includes, on the upper side the catheter 200, a bridge portion 214a that protrudes beyond the catheter 200 from the arrow B2 side to the arrow B1 side and an L-shaped first hook portion 214b (the holding portion 116) that is bent to the arrow C2 side from an end of the bridge portion 214a on the arrow B1 side and protrudes to the arrow B1 side. Further, a recessed portion 214c recessed to the arrow B2 side is provided in the first support member 214 on the arrow C2 side of the bridge portion 214a. On the other hand, the second support member 216 includes, on the lower side of the catheter 200, a bridge portion 216a that protrudes beyond the catheter 200 from the arrow B1 side to the arrow B2 side, and a second hook portion 216b that is bent to the arrow C1 side from an end of the bridge portion 216a on the arrow B2 side and protrudes to the arrow B2 side. Further, a recessed portion 216c (the insertion target portion 128) is provided on the arrow B1 side in the second support member 216 on the arrow C1 side of the bridge portion 216a.

The first hook portion 214b of the first support member 214 is held and fitted to the second support member 216 by being inserted into the recessed portion 216c of the second support member 216. The second hook portion 216b of the second support member 216 is held and fitted to the first support member 214 by being inserted into the recessed portion 214c of the first support member 214. As a result, in the hook mechanism 217, the first support member 214 and the second support member 216 firmly support each other in the arrow B direction and the arrow C direction, thereby forming a quadrangular support hole 218 surrounded by the first bridge portion 214a, the first hook portion 214b, the second bridge portion 216a, and the second hook portion 216b. The catheter 200 inserted into the support hole 218 is firmly supported on the distal side of the grip 18 by the hook mechanism 217.

In addition, a proximal end of the second support member 216 has a connection protrusion 216d connected to the grip 210, and the connection protrusion 216d has a round hole 216d1 formed to penetrate in the arrow C direction and pivotally supported by a shaft pin 210a. On the other hand, a proximal end of the first support member 214 also has a connection protrusion 214d connected to the grip 210, and the connection protrusion 214d protrudes to the arrow A2 side longer than the connection protrusion 216d. The connection protrusion 214d is provided with a long hole 214d1 that is formed to penetrate in the arrow C direction and is pivotally supported by the shaft pin 210a. Further, the first support member 214 includes an abutment portion 220 on which the distal end of the catheter hub 20 abuts prior to the second support member 216 when the catheter hub 20 relatively advances with respect to each of the grips 210.

The catheter assembly 10A according to the second embodiment is basically configured as described above, and operations thereof will be described hereinafter. During use, the user of the catheter assembly 10A grips the grip 210 and punctures the treatment target P.

At this time, the first and second support members 214 and 216 are held and engaged with each other to support the catheter 200 (multi-structure needle 204) (see FIG. 10A). Therefore, even if upward and lateral forces are applied to the support structure 212 from the multi-structure needle 204 in contact with the treatment target P, the support structure 212 is not deformed in the upward and lateral directions, and the deflection of the multi-structure needle 204 can be suppressed. As a result, the catheter assembly 10A can favorably insert the catheter 200 (multi-structure needle 204) into the body.

After the puncture, the user advances the catheter hub 206 in the distal direction (to the arrow A1 side) to allow the catheter 200 to enter a blood vessel. During the advancement, the distal end of the catheter hub 206 first abuts on the abutment portion 220 of the first support member 214 and pushes out the first support member 214 in the distal direction (see FIG. 10B). Since the long hole 214d1 is pivotally supported by the shaft pin 210a, the first support member 214 can move in the distal direction relative to the second support member 216 by being pushed out. That is, the first support member 214 and the second support member 216 support each other in the arrow B direction and the arrow C direction by the hook mechanism 217, and the first support member 214 can be easily moved in the arrow A direction.

This movement of the first support member 214 causes the release of the engagement of the hook mechanism 217 between the first support member 214 and the second support member 216 in the catheter assembly 10A. When the engagement is released, the first support member 214 and the second support member 216 rotate to be separated from each other with the shaft pin 210a as a base point, thereby allowing the advancement of the catheter hub 206. As a result, the catheter assembly 10A can easily separate the catheter 200 and the catheter hub 206 from the inner needle 202, the inner needle hub 208, the grip 210, and the support structure 212.

Technical ideas and effects that can be grasped from the above-described embodiment are described as follows.

The catheter assembly 10 or 10A according to an aspect of the present invention includes: the catheter 12 or 200; the catheter hub 20 or 206 fixed to the catheter 12 or 200; the inner needle 14 or 202 inserted through the catheter 12 or 200; the grip 18 or 210 that fixes and holds the inner needle 14 or 202; the first support portion 62 configured as a member separate from the grip 18 or 210 and capable of supporting the outer side of the catheter 12 or 200; and the second support portion 121 that is configured as a member separate from the grip 18 or 210, has at least a part being located at a position facing the first support portion 62, and is capable of supporting the outer side of the catheter 12 or 200, the first support portion 62 and the second support portion 121 being directly held to be releasable from each other.

According to the above description, the catheter assembly 10 or 10A includes the first and second support portions 62 and 121 that are directly held with each other, so that the catheter 12 or 200 can be more reliably supported. Therefore, the catheter assembly 10 or 10A can suppress the deflection of the catheter 12 or 200 and the inner needle 14 or 202 at the time of puncturing the treatment target P, and can favorably implement the puncture with the catheter 12 or 200 and the inner needle 14 or 202 performed by the user. In addition, since the first and second support portions 62 and 121 are configured as the members separate from the grip 18 or 210, the grip 18 or 210 has a wider opening portion for delivering the catheter hub 20 or 206, and easily delivers the catheter 12 or 200 from the catheter hub 20 or 206. That is, the catheter assembly 10 or 10A can further improve the operability of the user.

In addition, the first support portion 62 is the operation unit 61 configured to operate the relative movement of the catheter 12 with respect to the grip 18. As a result, the operation unit 61 held by the second support portion 121 is not separated from the second support portion 121 at the time of puncture, and the support of the catheter 12 can be favorably maintained.

In addition, the operation unit 61 is slidable along the extending direction of the catheter 12 in a state of being held by the second support portion 121, and is not separable in the direction in which the operation unit 61 and the second support portion 121 overlap each other. As a result, the catheter assembly 10 can more firmly support the catheter 12 sandwiched in the direction in which the operation unit 61 and the second support portion 121 overlap each other. In addition, when the user operates the operation unit 61 along the extending direction of the catheter 12, the holding of the operation unit 61 and the second support portion 121 can be easily released.

In addition, at least a part of operation unit 61 is engaged with grip 18 not to be detachable in the direction in which operation unit 61 and second support portion 121 overlap each other. As a result, the operation unit 61 does not come out of the grip 18 at an accommodating position of the grip 18, and thus, it is possible to more reliably inhibit deformation of the operation unit 61 and the deflection of the catheter 12 accompanying the deformation.

In addition, the operation unit 61 has the reinforcing portion (reinforcing piece 119), which inhibits deflection in a direction away from the catheter 12, on the distal side. As a result, the operation unit 61 can more reliably suppress deformation of a portion engaged with the second support portion 121.

In addition, the first support portion 62 includes: an extending portion (the operation plate portion 63 and the first support member 214) extending along the catheter 12; a protruding portion (the lower protruding block 115 and the bridge portion 214a) that protrudes from the extending portion beyond the catheter 12; and the holding portion 116 (first hook portion 214b) that extends from the protruding portion toward the second support portion 121 and holds the second support portion 121. As a result, the catheter assembly 10 or 10A can easily hold the holding portion 116 with respect to the second support portion 121.

In addition, the second support portion 121 includes the insertion target portion 128 that allows slidable insertion of the holding portion 116 to hold the holding portion 116. As a result, the catheter assembly 10 can easily hold the first support portion 62 and the second support portion 121 by the holding portion 116 inserted into the insertion target portion 128.

In addition, the holding portion 116 and the insertion target portion 128 extend along the extending direction of the catheter 12. As a result, the catheter assembly 10 can smoothly separate the holding portion 116 from the insertion target portion 128 by moving the first support portion 62 in the extending direction of the catheter 12 after puncture with the catheter 12 and the inner needle 14.

In addition, the protruding portion (the lower protruding block 115) and the holding portion 116 are provided as a pair with the catheter 12 interposed therebetween. As a result, the first support portion 62 and the second support portion 121 can more stably form the mutually holding state around the catheter 12.

In addition, at least one of the first support portion 62 and the second support portion 121 can support the catheter 12 or 200 on the front side of the distal end of the grip 18 or 210. As a result, the catheter assembly 10 or 10A can reliably support the catheter 12 or 200 and the inner needle 14 or 202 even on the front side of the grip 18, and can favorably suppress the deflection thereof.

What is claimed is:

1. A catheter assembly comprising:
a catheter;
a catheter hub fixed to the catheter;
an inner needle inserted through the catheter;
a grip fixed to and holding the inner needle;
a first support portion, which is a member separate from the grip and is configured to support an outer side of the catheter; and
a second support portion, which is a member separate from the grip, has at least a part that is located at a position facing the first support portion, and is configured to support the outer side of the catheter;
wherein the first support portion and the second support portion are releasably held with each other.

2. The catheter assembly according to claim 1, wherein:
the first support portion is an operation unit configured to be operated to cause relative movement of the catheter with respect to the grip.

3. The catheter assembly according to claim 2, wherein:
in a state of being held by the second support portion, the operation unit is slidable along an extending direction of the catheter, and the operation unit is not separable from the second support unit in a direction in which the operation unit and the second support portion overlap each other.

4. The catheter assembly according to claim 2, wherein:
at least a part of the operation unit is engaged with the grip such that the operation unit is not detachable from the grip in a direction in which the operation unit and the second support portion overlap each other.

5. The catheter assembly according to claim 2, wherein:
the operation unit comprises a reinforcing portion on a distal side, the reinforcing portion inhibiting deflection of the operation unit in a direction away from the catheter.

6. The catheter assembly according to claim 1, wherein:
the first support portion comprises:
an extending portion extending along the catheter,
a protruding portion that protrudes from the extending portion beyond the catheter, and
a holding portion that extends from the protruding portion toward the second support portion and holds the second support portion.

7. The catheter assembly according to claim 6, wherein:
the second support portion includes an insertion target portion into which the holding portion is slidably insertable such that the insertion target portion holds the holding portion.

8. The catheter assembly according to claim 7, wherein:
the holding portion and the insertion target portion extend along an extending direction of the catheter.

9. The catheter assembly according to claim 6, wherein:
the protruding portions and the holding portions are each provided in pairs with the catheter between.

10. The catheter assembly according to claim 1, wherein:
at least one of the first support portion and the second support portion is configured to support the catheter at a location frontward of a distal end of the grip.

11. A catheter assembly comprising:
a catheter;
a catheter hub fixed to the catheter;
an inner needle inserted through the catheter;
a grip fixed to and holding the inner needle;
a catheter operation member, which is a member separate from the grip and is configured to support an outer side of the catheter, the catheter operation member comprising:
an operation plate portion extending in an axial direction of the catheter, and
a hub engagement portion connected to a proximal end of the operation plate portion and engaged with the catheter hub; and
a lower support member that is rotatable attached to the grip, has at least a part that is located at a position facing the catheter operation member, and is configured to support the outer side of the catheter, wherein the lower support member comprises:
a shaft extending in a direction in which the catheter operation member and the lower support member overlap, and
a supporting body portion protruding in a direction orthogonal to the direction in which the shaft extends;
wherein the catheter operation member and the lower support member are releasably engaged with each other.

12. A catheter assembly comprising:
a catheter;
a catheter hub fixed to the catheter;
an inner needle inserted through the catheter;
a grip fixed to and holding the inner needle;
a first support member connected to the grip on a first side and configured to support an outer side of the catheter, wherein the first support member comprises:
a first bridge portion that protrudes beyond the catheter in a first direction, and
a first hook portion that is bent downward from an end of the bridge portion and protrudes in the first direction; and
a second support member connected to the grip on a second side, wherein the second support member has at least a part that is located at a position facing the first support member and is configured to support the outer side of the catheter, and wherein the second support member comprises:
a second bridge portion that protrudes beyond the catheter in a second direction, and
a second hook portion that is bent upward from an end of the bridge portion and protrudes in the second direction;
wherein the first member portion and the second support member are releasably engaged with each other.
